(19) United States
(12) Patent Application Publication
(10) Pub. No.: US 2014/0302156 A1
(43) Pub. Date: Oct. 9, 2014

(54) RIGID SHAPED ARTICLE FOR THE TREATMENT OF ITCHING

(71) Applicant: Norbert Beermann, Munchen (DE)

(72) Inventor: Norbert Beermann, Munchen (DE)

(21) Appl. No.: 14/240,877

(22) PCT Filed: Aug. 25, 2011
(86) PCT No.: PCT/EP2011/064635

§ 371 Date: May 2, 2014

(51) Int. Cl. A61K 35/64; A61K 9/14; A61K 9/00

(52) U.S. Cl. CPC A61K 35/644; A61K 9/0014; A61K 9/143
USPC 424/498; 424/537; 424/725; 514/762; 514/547

(57) ABSTRACT

The invention concerns a rigid shaped article essentially consisting of a plurality of mutually adhering grains of sand provided with a coating consisting of wax, for the elimination or relief of itching. In a particular embodiment of the invention, the wax essentially consists of beeswax.

### FIELD OF THE INVENTION

The invention relates to a rigid shaped article which essentially consists of a plurality of mutually adhering grains of sand with a coating consisting of wax, for the elimination or relief of itching. In a particular embodiment of the invention, the wax consists essentially of beeswax.

### PRIOR ART

Itching, the medical term for which is pruritus, is an unpleasant sensitivity of the skin which provokes scratching. Basically, itching can be triggered by a variety of mediators such as, for example, medication, food, allergies, plant or insect poisons, or damage to the skin. In many cases, the itching is a symptom of a disease. Chronic forms of pruritus are often resistant to therapy.

Safe medication which helps in the long term against itching is unknown. Opioid antagonists such as Naltrexon®, for example, may have some success. In local itching, cooling generally helps. Warm water may also be of some help, but this leads to dry skin which can then itself give rise to itching. Light therapy with UVB radiation can also in some cases result in relief from the itching. Known prior art means and methods for the treatment of itching are unsatisfactory, however, since they usually provide only little or temporary success; the itching however, may reappear within a short period, in particular with treatment using medication, which is associated with strong or unwanted side effects.

Thus, there is a need for improved means for the treatment, i.e. elimination or relief, of itching, in particular for means which result in a long-term, complete elimination or relief from itching and which is free of side effects.

DE 19 615 896 C2 discloses a rigid sand article consisting of a plurality of mutually adhering grains of sand which are provided with a coating consisting of wax. On rubbing the sand article, discrete or agglomerated grains of sand are obtained which are provided with a coating consisting of wax. The use of the sand article as a sand scrub for skin massage is disclosed.

### AIM OF THE INVENTION

The aim of the invention is to provide a means which relieves or eliminates itching. A further aim of the present invention is to provide a means which relieves or eliminates itching and wherein the relief or elimination from itching is long-lasting and free from unwanted side effects. Further, the means should be easy to handle, free of substances which are harmful to health and thus also be suitable for children.

### SUMMARY OF THE INVENTION

Surprisingly, the present invention has established that the sand article known from DE 19 615 896 C2 (hereinafter termed a shaped article or rigid shaped article) is suitable for relieving or eliminating itching. The aim of the invention is thus accomplished by using a rigid shaped article consisting of a plurality of mutually adhering grains of sand with a coating consisting of wax for the treatment of itching.

Thus, the subject matter of the invention is a rigid shaped article as claimed in claim 1 for the treatment of itching. Advantageous or particularly appropriate embodiments of the subject matter of the application are defined in the dependent claims. Furthermore, the invention encompasses the use of the rigid shaped article for the treatment of itching.

### DETAILED DESCRIPTION OF THE INVENTION

In accordance with the invention, it has surprisingly been shown that a product consisting only of grains of sand and wax, in particular beeswax, wherein the wax completely or partially coats the grains of sand, can be used in diverse ways for the treatment, i.e. elimination or relief, of itching.

There is no particular limitation on the wax used in accordance with the invention which coats the plurality of grains of sand. Thus, the wax may be a natural wax, i.e. animal or vegetable wax, a mineral wax, a petrochemical wax or a synthetic or chemically modified wax. The wax may also be a mixture of any waxes, for example a mixture of one or more animal or vegetable waxes.

Examples of vegetable waxes are candelilla wax, carnauba wax, Japan wax, esparto grass wax, cork wax, guaruma wax, rice oil wax, sugar cane wax, ouricury wax and montan wax. Examples of animal waxes are beeswax, shellac wax, wool wax, spermaceti and lanolin. Examples of mineral waxes are ceresin and ozokerite and hard paraffin. Examples of petrochemical waxes are petrolatum, paraffin waxes, stearin waxes and microwaxes. Examples of chemically modified waxes are hard waxes, such as montan ester waxes, sasol waxes and hydrated jojoba waxes. Examples of synthetic waxes are polyalkylene waxes and polyethylene glycol waxes. According to the invention, beeswax is preferred.

The wax used in the invention is normally workable at room temperature, melts above 40° C. without decomposition, is of relatively low viscosity above the melting temperature zone and is not ropy. As a rule, between approximately 50° C. and 90° C., waxes pass into the molten, low viscosity state. Generally, beeswax is liquid at temperatures of 62° C. to 65° C.

There is no particular limitation on the sand contained in the shaped article of the invention. The grains of sand usually have a diameter of 0.06 to 3 mm and are described as a function of the grain size as fine sand (equivalent diameter usually 0.06 to 0.2 mm), medium sand (equivalent diameter usually 0.2 to 0.63 mm) or coarse sand (equivalent diameter usually 0.63 to 3.0 mm). Preferably, sand with a diameter of 0.1 to 0.5 mm is used. As an example, dune sand from the seashore can be used with grains which usually have a diameter of approximately 1 mm or less and which can if necessary be ground or screened to the desired grain size.

The suitable weight ratio of sand to wax is approximately 1:1 to 25:1, preferably 4:1 to 12:1, particularly preferably 6:1 to 10:1.

The grains of sand contained in the rigid shaped article are provided with a complete or partial coating consisting of wax. Preferably, the grains of sand contained in the shaped article of the invention are completely coated with wax.

The rigid shaped article of the invention essentially consists of a plurality of grains of sand coated with wax. The term “essentially” means that the rigid shaped article preferably exclusively consists of grains of sand coated with wax. In a further embodiment, the shaped article of the invention may, however, also contain small quantities, preferably <10% by weight, particular preferably <5% by weight of additives with respect to the total weight of the shaped article of the invention. Examples of additives which are suitable for use in the present invention are cosmetic additives such as skin care substances or antiperspirants, fragrances, colorants, consistency enhancers, essential and/or vegetable oils, vitamins and medically active ingredients.

The manufacture of the rigid shaped article of the invention is described in the prior art and in DE 19 615 896 C2. At its simplest, the manufacture involves heating the grains of sand and wax separately from each other to a temperature of 50-90° C., bringing the heated grains of sand and the heated liquid wax together and allowing the mass obtained to cool and solidify. This method means that the sand can be fixed without the use of synthetic resins and any solvents, simply by adding energy.

The rigid shaped article of the invention is preferably in the form of a stick, particularly preferably with a diameter of 1 to 3 cm and/or a length of 3 to 10 cm. if the diameter were to be less than 1 cm, the rigid shaped article would tend to break up; if the diameter were to be more than 3 cm, the rigid shaped article would become difficult to handle. In a further embodiment, the rigid shaped article is in the form of a cuboid or sphere, preferably with a diameter of 5 to 10 cm and/or a length of 5 to 10 cm. The desired shape of the shaped article of the invention may, for example, be obtained using the method described above and bringing the heated grains of sand and the liquid wax together in appropriate moulds, or pressing the mass obtained into appropriate moulds prior to cooling. Alternatively, the desired shape of the shaped article can be obtained by stamping or cutting the solidified mass, for example.

In accordance with the present invention, the rigid shaped article is suitable for the treatment, i.e. elimination or relief, of all types of itching. The itching in this case might be a side effect of skin diseases, for example eczema or fungal infections. Other possible triggers of itching are disorders such as allergies caused, for example, by animal hair, pollen, mites, chemicals or foods, neurodermatitis, diabetes or psoriasis. Itching may also be triggered by degenerative skin changes associated with aging which can result in dry or cracked skin. Examples of other possible causes of itching are irritation of the skin brought about, for example, by mechanical irritation such as shaving the legs or arms. Further causes of itching are insect toxins such as delivered by insect bites and stings such as mosquito bites, horsefly bites, bee stings or wasp stings.

In order to eliminate or relieve itching, the rigid shaped article of the invention is brought into contact with the site of the itch and preferably repeatedly rubbed over the site of the itch with a light pressure. In particular, the multiple to and fro movement or rubbing of the rigid shaped article over the site of the itch gradually takes the itching away. The rigid shaped article can be rubbed over the site of the itch for a period of more than one minute, for example 1 to 15 min, preferably 2-10 min. Usually, after a few minutes rubbing, the itching has completely disappeared. Preferably, when the treatment is finished, i.e. when rubbing with the rigid shaped article is finished, the itching does not reappear. Normally, the itching does not reappear even after a period of 24 hours following the end of the treatment. However, if the itching recurs, the treatment as described above can be repeated any number of times without harmful side effects.

A positive side effect of the treatment described above is a pleasant massaged effect. In particular, when the rigid shaped article is rubbed over the site of the itch using an increased pressure, then a massage effect can be obtained, increasing the blood flow of the treated skin. The increased blood flow can also promote the healing process, for example if the itching is caused by an inflammatory disorder.

Particularly advantageous effects and actions are obtained when the grains of sand contained in the shaped article of the invention are essentially coated with beeswax. The term “essentially” means that the grains of sand are preferably coated exclusively with beeswax, but also small quantities of other waxes, such as cheaper stearin or paraffin waxes, may be present. Here, the small quantities are preferably <20% by weight, particularly preferably <5% by weight with respect to the total weight of the wax. If the shaped article is manufactured with beeswax, each individual grain of sand has a complete or partial beeswax coating of the grain of sand, generally on a micrometric scale. The advantage of beeswax over other waxes is that beeswax is a natural product. Thus, beeswax is an environmentally and resource-friendly product which is generally free from additives which are chemicals and potentially hazardous to health.

Prior to rubbing the rigid shaped article of the present invention, the shaped article is pressed onto the skin to a greater or lesser extent. By means of the to and fro movement of the shaped article, under light or heavy pressure, over the site of the itching, the beeswax coating the grains of sand is released completely or partially from the grain of sand and thus can be deposited in thin layers on the skin and penetrate partially into the skin. Since natural beeswax regularly contains traces of healing substances such as propolis, for example, these substances can penetrate the skin when the shaped article is used and thus their positive effects can develop. In addition, propolis has proven antibiotic, antiviral and antifungal effects. The positive effects of propolis are in particular due to prenylated flavonoids, which have an antioxidant action. When beeswax is used, then, the positive effects described above can be produced in an advantageous manner.

By using the rigid shaped article as described above, the wax, in particular beeswax, can be applied to the skin without major technological aids and can be dosed extremely sparingly. The gliding and lubricating effect of the wax achieves this without rubbing hard, so that the site of the itching is soothed and is no longer irritated. Even when used with a high pressure, when a massage effect is to be produced at the same time, the skin is not irritated or damaged. In particular, an application which is kind to the skin is free from harmful side effects and the ease of use means that the shaped article of the invention is not only suitable for adults, but is also suitable for children.

Without wishing to be bound by any one theory, rubbing off the wax from the grains of sand onto the skin occurs so easily because on rubbing, a little frictional heat is produced, both on the skin to be treated and also on the grains of sand. Because of this simultaneous heating of the grains of sand surrounded by wax and the skin slightly warmed by rubbing, the wax remains stuck to the skin and can penetrate the skin. The grains of sand partially detach from the shaped article so that gradually, the grains of sand roll off the shaped article. Such an effect could not occur with a lump of pure wax such as a lump of pure beeswax, i.e. without the simultaneous presence of the grains of sand coated with wax. In this regard, rubbing would not operate properly as not enough friction would be generated, and thus neither the skin nor the lumps of wax could be warmed up. The maximum effect would be lubrication of the skin.

Particularly good results in the treatment of itching are obtained when the rigid shaped article is at least partially roughened prior to bringing it into contact with the site of the itching. The site of the itching is then brought into contact with the roughened side or surface of the shaped article as described above. Roughening is preferably carried out with a sharp or pointed object such as a knife, for example. By means of roughening, even a light pressure of the shaped article on the skin rubs grains of sand off so that the user finds that treatment of the site of the itching with the appropriately roughened shaped article can be accomplished particularly easily, i.e. does not require a great deal of force. Using it is thus particularly pleasant and can be effective in a shorter time compared with using the non-roughened shaped article.

The invention will now be illustrated using the following examples.

### EXAMPLES

### Example 1

### Elimination of Itching Caused by Allergy to Pollen

A female subject suffering from an allergy to pollen experienced severe itching following contact with various grasses at regions which had come into contact with the grasses. After using a rigid shaped article which consisted of sand and beeswax in a weight ratio of 8:1 by rubbing the itching regions for several minutes, the itching was alleviated and disappeared completely after approximately 10 to 15 minutes treatment. Even in the following 24 hours, the itching did not reappear. Using the shaped article of the present invention thus resulted in complete elimination of the itching.

### Example 2

### Elimination of Itching Caused by Food Allergy

A male subject suffering from a food allergy experienced severe itching at different regions on the skin after drinking various fruit juices. Rubbing with a rigid shaped article which consisted of sand and beeswax in a weight ratio of 8:1 for a period of 15 to 20 minutes resulted in complete disappearance of the itching. Even in the following 24 hours, the itching did not reappear. Using the shaped article of the present invention thus resulted in complete elimination of the itching.

### Example 3

### Elimination of Itching Caused by Insect Bite

After suffering several insect bites on various parts of the body, a male subject developed severe itching at the bite regions. After rubbing a rigid shaped article which consisted of sand and beeswax in a weight ratio of 8:1, the itching was alleviated and disappeared completely after 15 minutes use. Even in the following 24 hours, the itching did not reappear at the bite regions. Using the shaped article of the present invention thus resulted in complete elimination of the itching.

### Example 4

### Elimination of Itching Caused by Mechanical Irritation of the Skin (Shaving)

After shaving her legs, a female subject developed a burning, itching feeling on the skin which had been irritated by the effect of shaving, visible by reddening of the skin. After rubbing over the shaved areas for a few minutes with a shaped article consisting of sand and beeswax in a weight ratio of 8:1, the subjective feeling of itching and burning disappeared completely. Using the rigid shaped article had thus soothed the irritated skin and resulted in complete disappearance of the burning and itching feeling.

### Example 5

### Elimination of Itching Caused by Diabetes

A male subject suffering from diabetes experienced severe itching in his feet after eating calorie-laden food. After rubbing over the itching regions for a few minutes with a shaped article consisting of sand and beeswax in a weight ratio of 8:1, the itching subsided and disappeared completely after 10 to 15 minutes. Even in the following 24 hours, the itching did not reappear at the bite regions. Using the shaped article thus resulted in complete elimination of the itching.

### Example 6

### Elimination of Itching Caused by Insect Bites

After suffering several insect bites, a female subject experienced severe itching at the bite regions. After a few minutes rubbing over the itching regions with a shaped article consisting of sand and beeswax in a weight ratio of 8:1, the itching subsided and disappeared completely after approximately 10 to 15 minutes. Even in the following 24 hours, the itching did not reappear at the bite regions. Using the shaped article thus resulted in complete elimination of the itching.

### Example 7

### Elimination of Itching Caused by Skin Allergy

A male subject suffering with a skin allergy experienced severe itching in the shoulder and back. After a few minutes rubbing over the itching regions with a shaped article consisting of sand and beeswax in a weight ratio of 8:1, the itching subsided and disappeared completely after approximately 10 to 15 minutes. Even in the following 24 hours, the itching did not reappear at the bite regions. Using the shaped article thus resulted in complete elimination of the itching.

### Example 8

### Elimination of Itching Caused by Insect Bites, in Children

Two children aged 10 and 12 years experienced severe itching at the bite regions after suffering insect bites. After rubbing themselves over the itching regions for several minutes with a shaped article consisting of sand and beeswax in a weight ratio of 8:1, the itching subsided and disappeared completely after approximately 10 to 15 minutes. Even in the following 24 hours, the itching did not reappear at the bite regions. Using the shaped article of the present invention thus resulted in complete elimination of the itching. This example further illustrates that the shaped article of the invention is easy to use and can readily be used by children.

The above examples show that using the rigid shaped article of the present invention results in relief and complete disappearance of itching brought about by a variety of causes. Furthermore, the rigid shaped article is easy to use, free from side effects and also from substances which are harmful to health, and is kind the skin. Thus, the shaped article is also suitable for use by children.

1. A rigid shaped article consisting essentially of a plurality of mutually adhering grains of sand provided with a coating consisting of wax, for the elimination or relief of itching.

2. The rigid shaped article as claimed in claim 1, wherein the wax is selected from animal wax, vegetable wax, mineral wax, petrochemical wax, synthetic or chemically modified wax or mixtures thereof.

3. The rigid shaped article as claimed in claim 1, wherein the wax is an animal wax.

4. The rigid shaped article as claimed in claim 1, wherein the wax is a vegetable wax.

5. The rigid shaped article as claimed in claim 1, wherein the wax is a petrochemical wax.

6. The rigid shaped article as claimed in claim 1, wherein the grains of sand coated with wax exhibit a weight ratio of sand to wax of 2:1 to 25:1.

7. The rigid shaped article as claimed in claim 1, wherein the grains of sand have a diameter of 0.06 to 3 mm.

8. The rigid shaped article as claimed in claim 1, wherein the itching is triggered by insect bites, allergies, neurodermatitis, skin damage and/or irritation, contact with nettles and/or diabetes.

9. The rigid shaped article as claimed in claim 1, wherein the shaped article is in the form of a stick.

10. The rigid shaped article as claimed in claim 1, wherein the rigid shaped article has a cuboid or spherical shape.

11. The rigid shaped article as claimed in claim 1 wherein, prior to bringing the shaped article into contact with the itching region, the shaped article is at least partially roughened.

12. The rigid shaped article of claim 3, wherein the animal wax is selected from the group of beeswax, shellac wax, wool wax, spermaceti, lanolin or mixtures thereof.

13. The rigid shaped article of claim 12, wherein the animal wax is beeswax.

14. The rigid shaped article of claim 4, wherein the vegetable wax is selected from the group of candelilla wax, carnauba wax, Japan wax, esparto grass wax, cork wax, guaruma wax, rice oil wax, sugar cane wax, ouricury wax, montan wax or mixtures thereof.

15. The rigid shaped article of claim 5, wherein the petrochemical wax is selected from the group of paraffin wax, stearin wax or mixtures thereof.

16. The rigid shaped article of claim 6, wherein the grains of sand coated with wax exhibit a weight ratio of sand to wax of 4:1 to 12:1.

17. The rigid shaped article of claim 16, wherein the grains of sand coated with wax exhibit a weight ratio of sand to wax of 6:1 to 10:1.

18. The rigid shaped article of claim 7, wherein the grains of sand have a diameter of 0.1 to 0.5 mm.

19. The rigid shaped article of claim 9, wherein the shaped article is in the form of a stick with a diameter of 1 to 3 cm and/or a length of 3 to 10 cm.

20. The rigid shaped article of claim 10, wherein the cuboid or spherical shaped rigid shaped article has a diameter of 5 to 10 cm and/or a length of 5 to 10 cm.
